(19) United States
(12) Patent Application Publication
(10) Pub. No.: US 2005/0053628 A1
(43) Pub. Date: Mar. 10, 2005

(54) Method and device for the classfication and cosmetic treatment of wrinkles

(76) Inventors: Daniela Montanari, Albignasego (Padova) (IT); Manuela Guglielmo, Vigonovo (Venezia) (IT)

Correspondence Address: HEDMAN & COSTIGAN P.C., 1185 AVENUE OF THE AMERICAS, NEW YORK, NY, 10036, US

(21) Appl. No.: 10/482,184

(22) PCT Filed: Jun. 26, 2002
(86) PCT No.: PCT/EP02/07114

(30) Foreign Application Priority Data
Jun. 29, 2001 (CH) 2001 1200/01

(51) Int. Cl. G06K 9/00; A61K 7/00

(52) U.S. Cl. 424/401; 382/128

(57) ABSTRACT

The present invention relates to a method for visuallly determining and classifying the degree of facial wrikledness of the human skin, allowing a cosmetic treatment proportional to the degree of wrinkledness observed. The present invention also relates to a device (1), similar to a slide ruler, which allows a simple and accurate identification and classifation of the degree of facial wrinkledness and the immediate association with a cosmetic treatment appropriate for the degree of wrinkledness observed.

The present invention relates to a method for the classification and cosmetic treatment of wrinkles.

In particular, the present invention relates to a method for determining and visually classifying the degree of wrinkledness of the human skin, allowing a cosmetic treatment appropriate for the degree of wrinkledness determined.

It is known that the aging of the skin is accompanied by the appearance of wrinkles, a kind of facial wrinkling which can apply to both the surface layers of the skin and also deeper layers, including the derma.

The origin of wrinkles is a complex phenomenon which can only partly be attributed to the age of the individuals. Various factors can in fact influence the formation of wrinkles, ranging from the general state of health to genetic predisposition, exposure to external agents harmful for the skin, so-called free radical generators.

It is known in fact that the skin is subject to an aging process whose precociousness is in direct relation to the exposure of the skin to aggressive external agents such as smog, frequent temperature changes, sunlight, ultraviolet rays of sun lamps, etc.

The presence of all these variables in skin aging processes and in the formation of wrinkles, makes it difficult to apply a correct cosmetic therapy only on the basis of the age of the person to be treated.

In spite of this, current anti-wrinkle cosmetic treatment is almost exclusively based on the age of the patient and does not therefore take due account of the other variables and effective degree of wrinkledness of the face to be treated.

Manufacturers of cosmetic products, in fact, sell anti-aging creams with increasing compositions of active principles only in relation to the age of the patient.

It has also been verified that a significant percentage of users tend to purchase anti-wrinkle products of the same line with the highest concentrations of active principles in the hope that they are more effective than those with a low concentration of active principles.

This behaviour however has its disadvantages of use and can also cause the appearance of side-effects on the skin.

The appearance of these local side-effects is much more probable and significant, from a dermatological point of view, when cosmetic creams containing high dosages of active principles with an exfoliating action (peeling effect), are inappropriately used.

In particular, it has been found that the use of creams based on active principles with an exfoliating/regenerating activity such as glycolic acid (fruit acids) or derivatives of vitamin A, at dosages not adequately proportional to the degree of wrinkledness to be treated, can give rise to localized burning, skin inflammations or excessive skin dryness.

In other words, owing to the difficulties so far observed in determining a correct and adequate treatment dosage, there is the risk of creating further damage to the skin tissues already subjected to aging processes.

The necessity is therefore felt for finding a method for classifying in advance the degree of individual skin aging or degree of face wrinkling, to enable an anti-wrinkle cosmetic treatment to be subsequently applied, in line with the degree of face wrinkledness determined.

One of the main objectives of the present invention consists in providing a method for the classification and cosmetic treatment of wrinkles which is reliable and substantially avoids or limits the appearance of the drawbacks observed in the known art.

Another objective of the present invention consists in providing a simple and immediate method for determining the degree of facial wrinkledness reached to allow a cosmetic therapy against skin aging to be defined.

Yet another objective consists in providing a method and device which enables a reliable and accurate self-assessment to be made of the degree of facial wrinkledness at low costs for the individual user.

These and other objectives which will be described in more detail hereunder, are achieved, according to a first aspect of the invention, by a device for classifying the degree of facial wrinkledness, comprising a supporting element on which photographic images or reproductions are arranged, portraying face wrinkling or wrinkles of varying depths, each of said photographic images or reproductions being associated with an anti-wrinkle product having a cosmetic anti-wrinkle activity in proportion to the degree of wrinkledness portrayed or reproduced in said images.

In the context of the present invention, the device of the invention is defined as a wrinkle classifying device as it allows wrinkles to be classified according to a wrinkledness scale.

In accordance with another aspect of the present invention, a method is provided for the classification and cosmetic treatment of wrinkles or face wrinkling, which comprises:
a preliminary visual observation phase of the wrinkles or face wrinkling of an individual,
subsequent visual comparison with a wrinkledness scale comprising a series of photographic images or reproductions arranged in increasing order of wrinkledness on a supporting structure, to determine the degree of wrinkledness closest to that observed for said individual,
association of the degree of wrinkledness determined in the previous phases with a cosmetic product having an anti-wrinkle activity proportional to the degree of wrinkledness determined in the previous phase,
application of a cosmetically effective quantity of said cosmetic product on the areas of the face subject to wrinkles.

The cosmetic method of the invention not only enables the classification of face wrinkles for the user, but also a simple association with a cosmetic product having an anti-wrinkle or anti-aging activity specifically studied for treating the degree of wrinkledness identified by the individual.

In the method of the invention, cosmetic preparations with increasing contents of active principles having an anti-wrinkle or anti-aging action are associated with greater degrees of extension and depth of face wrinkling.

In accordance with a preferred embodiment of the method of the invention, the product RUGASTIR produced by LABO, under the Swiss trade-mark license of Cosprophar Labo, is used as cosmetic product with an anti-wrinkle action.

In the wrinkle classifier of the invention, six female human faces affected by increasing degrees of wrinkledness are preferably portrayed in the photographic images.

The images illustrate faces of individuals in which the first corresponds to a first degree of wrinkledness distinguished by initial and slight wrinkles, a second degree with moderate and fine wrinkles, a third degree with widely-spread, medium wrinkles, a fourth degree with medium/deep wrinkles, a fifth with abundant, deep wrinkles and a sixth degree marked by numerous and very deep wrinkles.

The use of four RUGASTIR products incorporating increasing quantities of anti-wrinkle active principle, with 2.5; 3.5; 4.5; 5.5 mg/g of active principle respectively, is preferably associated with the four degrees of wrinkledness. The active principle advantageously consists of a biological reactor RB 160.

Further characteristics and advantages of the invention will be more evident from the description of a classification device of the degree of wrinkledness of a human face, illustrated in the enclosed drawings and provided for indicative and non-limiting purposes, wherein:

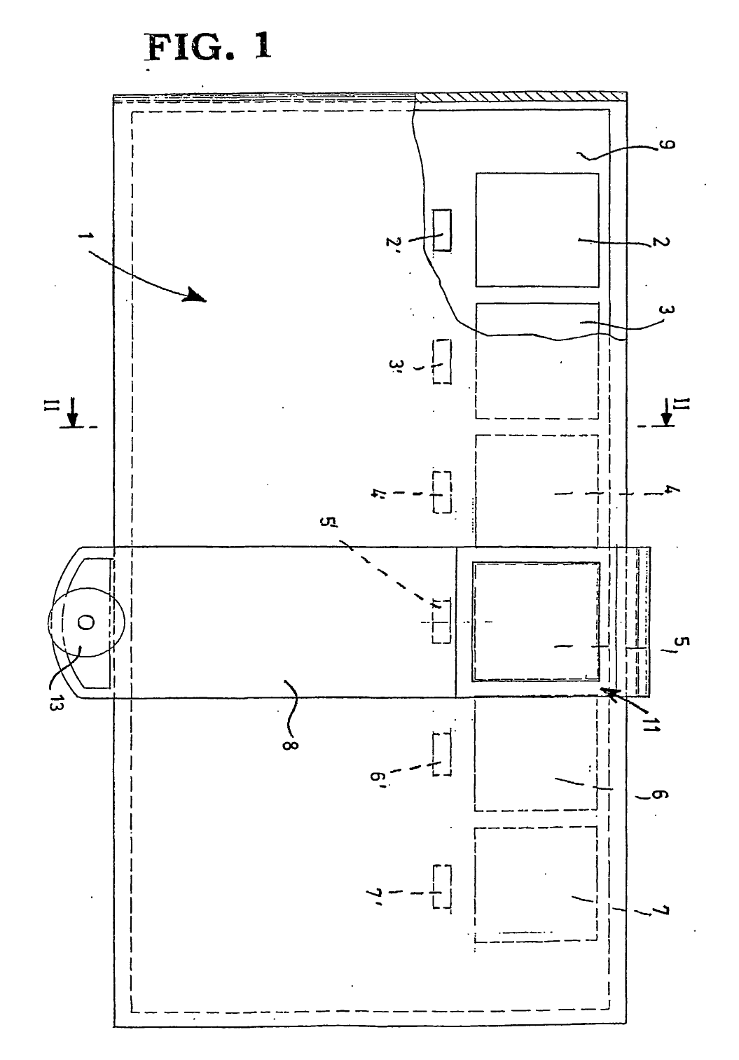

FIG. 1 is a plan view from above of the classification device of wrinkles,

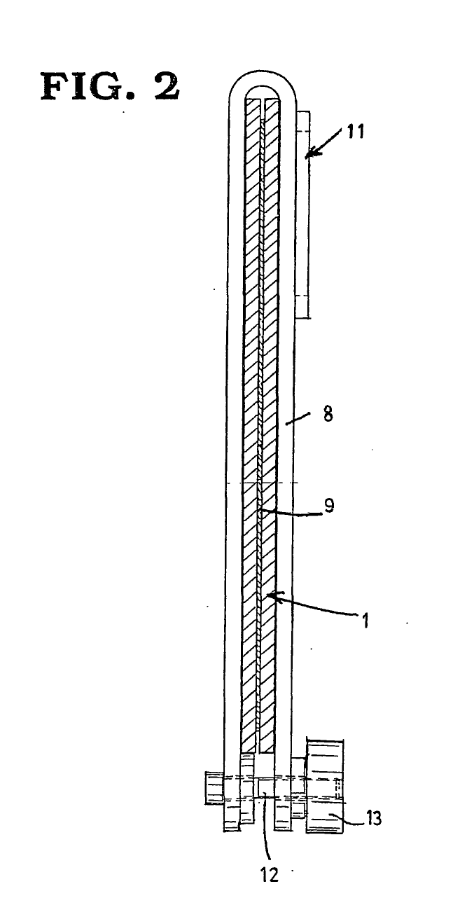

FIG. 2 is a longitudinal section of the device illustrated in FIG. 1.

With reference to the above figures, the classification device of wrinkledness comprises a supporting element on which photographic images or reproductions are arranged, on an increasing scale, portraying faces of people subject to the presence skin wrinkles of increasing gravity, starting from simple initial wrinkles of a slight degree to extremely numerous and deep wrinkles.

In particular, as illustrated in FIG. 1, the supporting element, indicated with reference number 1, comprises a rectangular-shaped element, advantageously made of Plexiglas, on which photographic images 2-7, side by side and portraying increasing degrees of wrinkledness, are arranged on a supporting sheet 9.

A cosmetic product (3′-6′) having an anti-wrinkle activity appropriate for the degree of wrinkledness of the image above, corresponds to each of four reproductions or images (3-6), univocally, for example in a position below said image. An increasing classification is thus obtained, of the degree of wrinkledness, starting from the image 2 and ending with the image portrayed with number 7. Compositions with an increasing anti-wrinkle activity or with increasing dosages of active principle correspond to four degrees of wrinkledness out of the six illustrated and consequently four images (3-6).

The device of the invention also advantageously comprises a sliding cursor 8, preferably equipped with a display window 11, situated on said supporting element 1. The cursor 8 slides horizontally along said supporting element 1 allowing a particular image (5 in FIG. 1) to be accurately identified, with which an anti-wrinkle cosmetic product (51 in FIG. 1), having an anti-wrinkle activity appropriate for the degree of wrinkledness illustrated in image 5, is associated. The positioning of the cursor 8 on the image 5 thus allows the degree of wrinkledness of the face portrayed to be classified and the most suitable cosmetic treatment determined.

FIG. 2 illustrates a transversal section of the device of the invention along the line II-II of FIG. 1.

FIG. 2 illustrates the cursor 8 which moves along the supporting structure 1 inside which there is a supporting sheet 9 on which the images 2-7 of FIG. 1 are arranged.

The cursor 8 is equipped with a window 11 to facilitate a correct and accurate positioning of the images 2-7 (not shown). The cursor 8 is constrained at one end to the supporting structure 1, and is secured by means of a screw 12 associated with a nut 13.

The device according to the invention fully satisfies the pre-established objectives, as it allows an accurate classification to be made of the degree of facial wrinkledness and the consequent univocal association with a cosmetic product having a composition specifically appropriate for the wrinkledness observed.

There are a wide variety of embodiments of the wrinkle classification device of the invention, one of these being illustrated in the enclosed drawings.

In practice, the materials used, and also the dimensions, provided they are compatible with the specific use described, can vary widely according to the specific demands.

1. Method for the classification and cosmetic treatment of wrinkles or face wrinkling, comprising:
a preliminary visual observation phase of the wrinkles or face wrinkling of an individual,
subsequent visual comparison with a wrinkledness scale comprising a series of photographic images or reproductions arranged in increasing order of wrinkledness on a supporting structure, to determine the degree of wrinkledness closest to that observed for said individual,
association of the degree of wrinkledness determined in the previous phases with a cosmetic product having an anti-wrinkle activity proportional to the degree of wrinkledness determined in the previous phase,
application of a cosmetically effective quantity of said cosmetic product on the areas of the face subject to wrinkles.

2. The method according to claim 1, wherein said wrinkledness scale comprises six degrees of facial wrinkledness.

3. The method according to claim 2, wherein an image of a human face with initial and slight wrinkles corresponds to a first degree of wrinkledness, an image of a face with moderate and fine wrinkles corresponds to a second degree, a face with widely-spread, medium wrinkles corresponds to a third degree, a face with medium/deep wrinkles corresponds to a fourth degree, a face with abundant, deep wrinkles corresponds to a fifth degree and a face with numerous and very deep wrinkles corresponds to a sixth degree.

4. The method according to one or more of the previous claims 13, wherein said cosmetic product comprises an skin anti-wrinkle or anti-aging active principle in increasing quantities and proportional to the degree of wrinkledness observed and classified.

5. The method according to one or more of the previous claims wherein said cosmetic product is RUGASTIR.

6. The method according to claim 5, wherein the local application of RUGASTIR 2.5 is associated with the second degree of wrinkledness; the application of RUGASTIR 3.5 with the third degree of wrinkledness; the application of RUGASTIR 4.5 with the fourth degree of wrinkledness and the application of RUGASTIR 5.5 with the fifth degree of wrinkledness.

7. The method according to claim 6 wherein said RUGASTIR incorporates a biological reactor RB160 with an anti-wrinkle and anti-aging activity of the skin.

8. The method according to one or more of the previous claims comprising the local application of a cosmetically effective quantity of said cosmetic product once or several times a day.

9. A device for classifying the degree of facial wrinkledness, comprising a supporting element on which photographic images or reproductions are arranged, which portray face wrinkling or wrinkles of varying depths, each of said photographic images or reproductions being associated with a product having an anti-wrinkle or anti-aging activity of the skin appropriate for the degree of wrinkledness illustrated or reproduced.

10. The device according to claim 9, wherein said supporting element is substantially rectangular-shaped, with said photographic images or reproductions arranged side by side.

11. The device according to claim 9 or 10, comprising a sliding cursor which moves along said supporting element to determine the photograph image or reproduction which is closest to the degree of face wrinkledness of the person to be treated.

12. The device according to one or more of claims 9-11 characterized in that it is made of transparent plastic material or glass.

13. The device according to claim 9, wherein said supporting element consists of a discoidal element on which said photographic images or reproductions are circumferentially arranged and the cosmetic products associated with each of said images are radially arranged.
